(19) United States
(12) Patent Application Publication
(10) Pub. No.: US 2008/0177252 A1
(43) Pub. Date: Jul. 24, 2008

(54) Wearable surgical drain and interchangeable reservoir

(75) Inventor: Frank Isik, Seattle, WA (US)

Correspondence Address: GRAYBEAL, JACKSON, HALEY LLP, 155 - 108TH AVENUE NE, SUITE 350, BELLEVUE, WA, 98004-5973, US

(73) Assignee: 3i Ventures LLC, Seattle, WA (US)

(21) Appl. No.: 12/008,938

(22) Filed: Jan. 14, 2008

Related U.S. Application Data
(60) Provisional application No. 60/881,881, filed on Jan. 22, 2007.

(51) Int. Cl. A61M 27/00

(52) U.S. Cl. 604/541

(57) ABSTRACT

A closed surgical drain assembly includes a base having an adhesive surface adapted to adhere to a patient's skin, the base including a reservoir retainer. The assembly further includes a removable reservoir adapted to be received and supported by the reservoir retainer of the base, and a drainage tube that provides fluid communication between the patient and the reservoir.

This application claims priority to U.S. Provisional Application Ser. No. 60/881,881, filed on Jan. 22, 2007, which is incorporated by reference.

### BACKGROUND

Following surgical dissection and/or tissue removal, a cavity known as “dead space” is created that can potentially fill with blood, plasma, and fluids. To prevent a seroma (body cavity fluid accumulation) in the “dead space”, the surgeon or interventionist often leaves a drain. The drain can stay in for days to weeks afterwards depending on the volume and characteristic of the drainage fluid. Typically these drains have a flexible narrow tube that is either perforated or slotted for the portion within the body cavity, which exits the skin where it is sutured. The drain continues from the skin as external tubing into a bulb reservoir collection device that hangs from the wound and is pinned to dressings or clothing. Whereas the intra-cavity component of the closed surgical drains are adequate (“Jackson-Pratt” or “Blake” drain designs), the portion of the drainage system after it exits the skin has several problems. The problems that plague current closed surgical drains are partly due to the long tube that connects to the external (bulb) reservoir.

Further, patient dissatisfaction is high with current surgical drains because of the inability to wear normal clothing. Patients simply do not know where to put the bulb reservoir. There is also the constant issue of what to do with the long drainage tube. Other problems include skin irritation and pulling at suture sites and leakage at skin insertion/exit sites.

In addition, the exposed drain tube requires frequent “stripping” to prevent clogging. Also, it is messy to empty the collection device for measuring purposes.

### SUMMARY OF THE INVENTION

In a first embodiment of the invention, a closed surgical drain assembly includes a base having an adhesive surface adapted to adhere to a patient's skin, the base including a reservoir retainer. The assembly further includes a removable reservoir adapted to be received and supported by the reservoir retainer of the base, and a drainage tube that provides fluid communication between the patient and the reservoir.

The reservoir retainer may comprise a pouch adapted to receive the reservoir. The pouch may be formed of compliant material.

The drainage tube may include a first drain tube portion adapted to extend proximally into the patient, a second drain tube portion communicating with the reservoir, and a coupling that couples the first drain tube portion with the second drain tube portion. The coupling may be a quick connect/disconnect coupling.

The first drain tube portion may be adapted to extend through an opening in the patient's skin and include a sealing flange that makes surface contact with the patient's skin about the opening. The adhesive surface of the base may be arranged to make adhesive contact with the first drain tube portion flange and with the patient's skin about the flange to form an adhesive supported seal about the opening.

The first drain tube portion may have an increasing diameter portion that increases in diameter in the direction of fluid flow towards the reservoir to prevent clogging of the drain. The increasing diameter portion may be immediately proximal of the first drain tube portion flange.

The reservoir has a distal end and may include a drainage port at its distal end. The reservoir may include fluid level indicia. The base may further include suture tabs adapted to receive suture and assist the adhesive surface in adhering the base to the patient's skin.

In another embodiment, a closed surgical drain assembly comprises a base adapted to be worn on a patient's skin, a reservoir supported by the base, and a drainage tube that provides fluid communication between the patient and the reservoir. The drainage tube includes an increasing diameter portion that increases in diameter in the direction of fluid flow, and is arranged to be beneath and immediately adjacent to the patient's skin.

The base may include a retainer for holding the reservoir. The retainer may comprise a pouch formed of compliant material.

The base may include an adhesive surface that adheres to the patient's skin. The drainage tube may include a first drain tube portion, including the increasing diameter portion, adapted to extend proximally into the patient, a second drain tube portion communicating with the reservoir, and a coupling that couples the first drain tube portion with the second drain tube portion. The coupling may be a quick connect/disconnect coupling.

The first drain tube portion may be adapted to extend through an opening in the patient's skin and may include a sealing flange that makes surface contact with the patient's skin about the opening. The base may have an adhesive surface arranged to make adhesive contact with the first drain tube portion flange and with the patient's skin about the flange to form an adhesive supported seal about the opening.

The features of the present invention which are believed to be novel are set forth with particularity in the appended claims. The invention, together with further features and advantages thereof, may best be understood by making reference to the following description taken in conjunction with the accompanying drawings, in the several figures of which like reference numerals identify identical elements, and wherein:

### DESCRIPTION OF THE DRAWINGS

FIG. 1 is a perspective view of a surgical drain assembly according to an embodiment of the invention;

FIG. 2 is a perspective representation of the drain and base unit of the surgical drain assembly of FIG. 1;

FIG. 3 is a top view of the surgical drain assembly of FIG. 1;

FIG. 4 is a top view of the surgical drain assembly of FIG. 1 showing the replaceable reservoir bag removed;

FIG. 5 is a front plan view of a closed surgical drain assembly according to another embodiment of the invention;

FIG. 6 is a rear plan view of the closed surgical drain assembly of FIG. 5; and

FIG. 7 is a partial side view showing details of the sealing of the drainage site with the closed surgical drain assembly of FIG. 5.

### DETAILED DESCRIPTION OF THE INVENTION

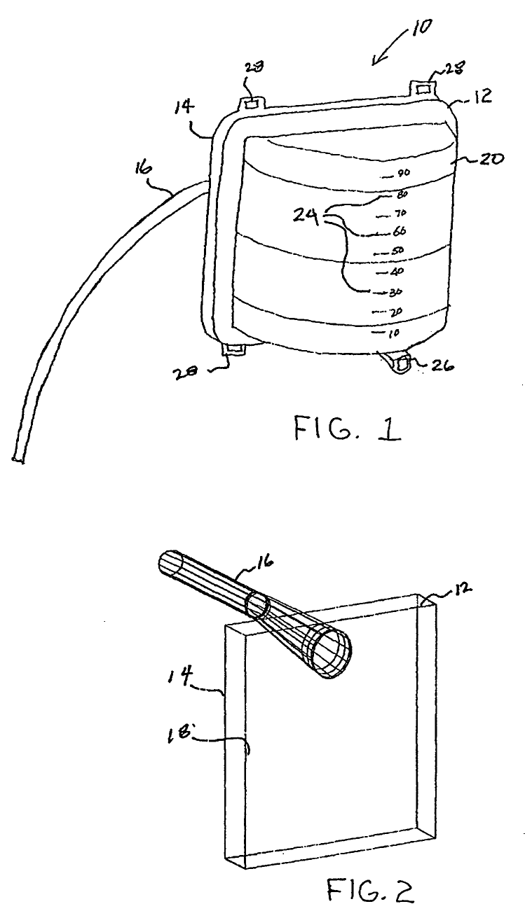

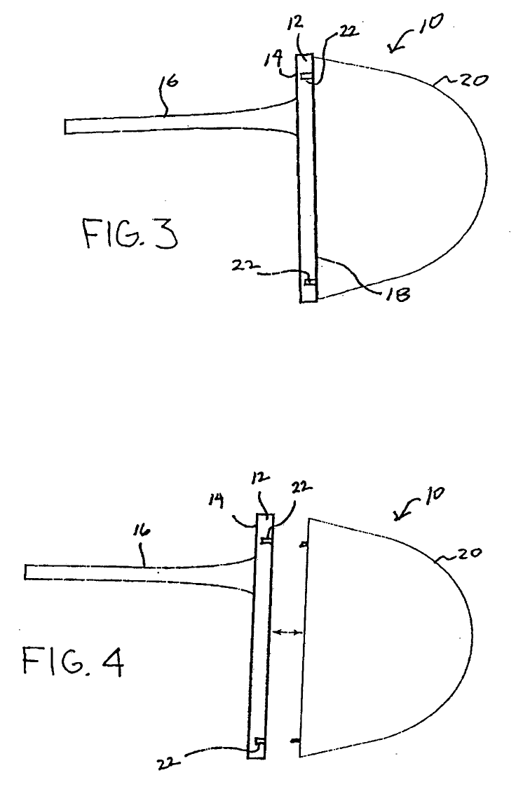

To circumvent the above noted problems, the present invention, in its various embodiments, provides improved closed surgical drain assemblies. The assembly 10 of FIGS. 1-4 is one such embodiment of the present invention. The closed surgical drain assembly there shown includes flexible anatomic base unit 12 that has a flat moldable, adherent surface 14 for skin adherence. The base 12 may thus be worn over or near the drainage tube insertion site. Reinforcement suture tabs 28 may be included for particularly difficult body sites. The anatomic base unit 12 is integrated with a tapered drain 16 exiting on the patient's skin to completely eliminate any external drainage tubing. On the outer-facing side, the anatomic base unit has receptacles 22 for attachment of a disposable snap-on collection reservoir bag 20.

The internal drain 16 is a tapered tube that widens in diameter as the drain leaves the body cavity and enters the reservoir base unit. This eliminates the need to periodically strip the drain and prevents clogging.

The disposable collection reservoir bag 20 may be removed from the anatomic base unit 12 when full, leaving the anatomic base unit 12 with drain 16 intact on the skin. A new disposable reservoir bag may then be easily snapped onto the moldable anatomic base unit. The disposable collection reservoir bag 20 is sealed but upon connection to base unit 12, auto-expands to apply negative pressure to the drain.

The sealed collection reservoir bag 20 may contain markers 24 indicating the amount of fluid within the reservoir, making measurement of the drainage amount easy and without mess. The patient or care providers do not have to manipulate the fluid and there is no risk of spilling or contact with the drainage fluid.

Alternatively or in addition, the reservoir bag 20 may contain a nipple valve 26 that self-seals to allow hook-up of a tubing-syringe combo to drain and measure amount or, a recharge packet that hooks into the nipple valve that can collapse the primary reservoir and drain the contents automatically. In some embodiments, large base units with attachable reservoirs could accommodate more than one drain for larger wounds that are difficult surgical management problems such as with open abdomens or complicated degloving injuries.

In one embodiment, the flexible anatomic unit and drain are integrated, but the reservoir is disposable and allows rapid re-attachment to the base anatomic unit. In another embodiment, the base anatomic unit, collection reservoir and drain are integrated as a single device. The catheter/drain portion that is within the body cavity can be of any configuration, with or without trocar.

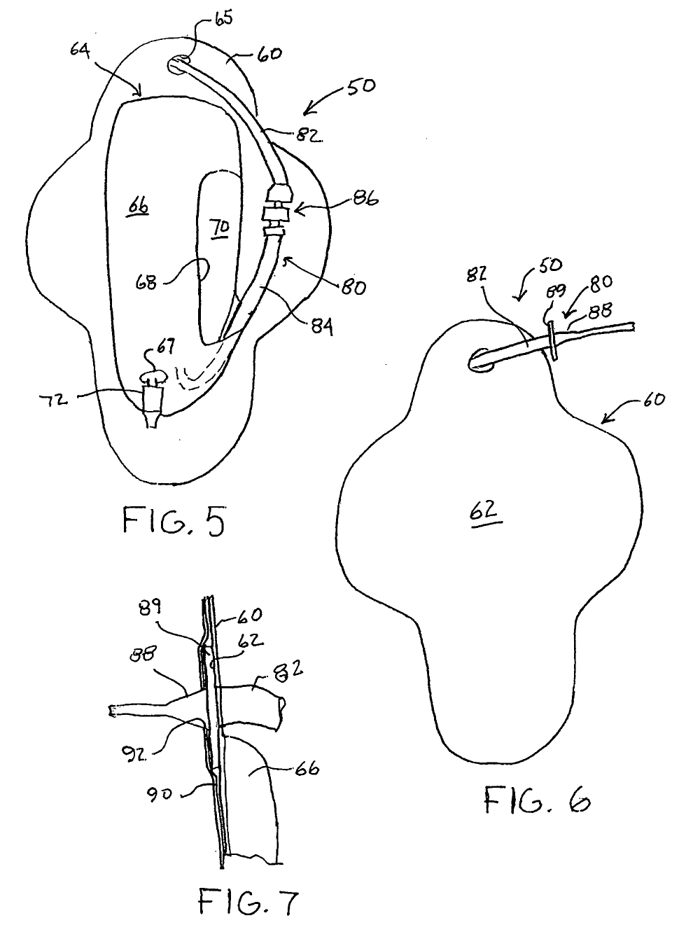

Referring now to FIGS. 5-7, they show another closed surgical drain assembly 50 according to a further embodiment of the invention. The assembly 50 generally comprises a base 60, a reservoir 70, and a drain tube assembly 80.

The base 60 is formed of flexible material to conform to the body contour of the patient. The base includes an adhesive coated rear surface 62 for adhering the base to the patient's skin. The adhesive surface 62 may initially be covered with a strippable cover that may be removed prior to deployment of the assembly 50.

The base 60 further includes a reservoir retainer to support and hold the reservoir 70. Here, the reservoir retainer 64 is a pouch 66. The pouch is preferably formed of a flexible compliant material as, for example, a flexible mesh material. The pouch includes a large opening 68 through which the reservoir 70 may be placed. The reservoir is preferably formed of a conformal elastic material so that it may be initially in a bundled configuration when placed into the pouch and then released to impose a negative pressure on the system as previously described.

While the retainer 64 in this embodiment comprises a pouch, other forms of retainers may be employed as well without departing from the present invention. Such other forms of retainers may comprise, for example and without limitation, straps, bands, Velcro, snaps, or tabs.

The reservoir includes a drain 72 at its distal end that projects through an opening 67 within the pouch 66. The drain permits the reservoir to be drained should it become full without a replacement being immediately available.

The drain assembly 80 includes a first drain tube 82 and a second drain tube 84. The second drain 84 tube is attached to the reservoir. The first drain tube 82 extends from within the patient at a drainage site. A coupling 86 couples the first and second drain tubes 82 and 84 together. The coupling may be, for example, a quick connect/disconnect coupling well know in the art. This enables a filled reservoir to be readily replaced with a new one without disturbing the drainage site.

As may best be seen in FIGS. 6 and 7, the first drain tube 82 includes a tapered section 88 that causes the tube 82 to increase in diameter in the direction of fluid flow from the patient to the reservoir 70. The tapered portion 88 terminates immediately adjacent and proximal to a sealing flange 89. The tapered portion 88 prevents the drain assembly 80 from clogging and eliminates the need to periodically strip the drain for cleaning.

The sealing flange 89 together with the adhesive surface 62 of the base 60 provides a sealed closed drainage system. As may be best seen in FIG. 7, the sealing flange 89 makes broad surface contact with the patient's skin about the opening 92 in the patient's skin 90 through which the first drain tube passes. The adhesive surface 62 of the base 60 makes broad covering surface contact with the sealing flange 89. Hence, the adhesive surface 62 of the base 60 is arranged to make adhesive contact with the first drain tube flange 89 and with the patient's skin 90 about the flange to form an adhesive supported seal about the opening 92.

From the foregoing, it may be seen that the present invention overcomes many of the problems experienced with prior art closed surgical drain systems. The drain assembly of the present invention may be worn by a patient in comfort and permits the patient to wear normal clothing. The sealing arrangement for the drainage sight minimizes leaks and completely seals the drainage sight. Still further, no long drain tube is required and drain tube stripping is eliminated by the anti-clogging configuration of the drain assembly. Still further, the reservoir and base may be made in any shape to conform with virtually every localized area of a patient's body. The reservoirs may be made in pre-fashioned shapes and sizes to this end.

While particular embodiments of the present invention have been shown and described, modifications may be made, and it is therefore intended in the appended claims to cover all such changes and modifications which fall within the true spirit and scope of the invention as defined by those claims.

What is claimed:

1. A closed surgical drain assembly comprising:
a base having an adhesive surface adapted to adhere to a patient's skin, the base including a reservoir retainer;
a removable reservoir adapted to be received and supported by the reservoir retainer of the base; and
a drainage tube that provides fluid communication between the patient and the reservoir.

2. The assembly of claim 1, wherein the reservoir retainer comprises a pouch adapted to receive the reservoir.

3. The assembly of claim 2, wherein the pouch is formed of compliant material.

4. The assembly of claim 1, wherein the drainage tube includes a first drain tube portion adapted to extend proximally into the patient, a second drain tube portion communicating with the reservoir, and a coupling that couples the first drain tube portion with the second drain tube portion.

5. The assembly of claim 4, wherein the coupling is a quick connect/disconnect coupling.

6. The assembly of claim 4, wherein the first drain tube portion is adapted to extend through an opening in the patient's skin and includes a sealing flange that makes surface contact with the patient's skin about the opening.

7. The assembly of claim 4, wherein the adhesive surface of the base is arranged to make adhesive contact with the first drain tube portion flange and with the patient's skin about the flange to form an adhesive supported seal about the opening.

8. The assembly of claim 6, wherein the first drain tube portion has an increasing diameter portion that increases in diameter in the direction of fluid flow towards the reservoir.

9. The assembly of claim 8, wherein the increasing diameter portion is immediately proximal of the first drain tube portion flange.

10. The assembly of claim 1, wherein the reservoir has a distal end, and wherein the reservoir includes a drainage port at its distal end.

11. The assembly of claim 1, wherein the reservoir includes fluid level indicia.

12. The assembly of claim 1, wherein the base further includes suture tabs adapted to receive suture and assist the adhesive surface in adhering the base to the patient's skin.

13. A closed surgical drain assembly comprising:
a base adapted to be worn on a patient's skin;
a reservoir supported by the base; and
a drainage tube that provides fluid communication between the patient and the reservoir, the drainage tube including an increasing diameter portion that increases in diameter in the direction of fluid flow, the increasing diameter portion arranged to be beneath and immediately adjacent to the patient's skin.

14. The assembly of claim 13, wherein the base includes a retainer for holding the reservoir.

15. The assembly of claim 14, wherein the retainer comprises a pouch formed of compliant material.

16. The assembly of claim 13, wherein the base includes an adhesive surface that adheres to the patient's skin.

17. The assembly of claim 13, wherein the drainage tube includes a first drain tube portion, including the increasing diameter portion, adapted to extend proximally into the patient, a second drain tube portion communicating with the reservoir, and a coupling that couples the first drain tube portion with the second drain tube portion.

18. The assembly of claim 17, wherein the coupling is a quick connect/disconnect coupling.

19. The assembly of claim 17, wherein the first drain tube portion is adapted to extend through an opening in the patient's skin and includes a sealing flange that makes surface contact with the patient's skin about the opening.

20. The assembly of claim 19, wherein the base has an adhesive surface arranged to make adhesive contact with the first drain tube portion flange and with the patient's skin about the flange to form an adhesive supported seal about the opening.
